(19) United States
(12) Patent Application Publication
(10) Pub. No.: US 2023/0099134 A1
(43) Pub. Date: Mar. 30, 2023

(54) NAIL MEASURING DEVICE FOR CUSTOMIZED NAIL PRODUCTS AND SERVICE PROVIDING METHOD USING THE NAIL MEASURING DEVICE

(71) Applicant: LUPIN COMPANY INC., Seoul (KR)

(72) Inventors: Ho Keun Kim, Gyeonggi-do (KR); Hwan Hee Koo, Gyeonggi-do (KR)

(21) Appl. No.: 17/527,780

(22) Filed: Nov. 16, 2021

(30) Foreign Application Priority Data
Sep. 27, 2021 (KR) 10-2021-0126983

(51) Int. Cl. A45D 31/00; G06Q 30/06; G06K 9/00; G06T 7/60; G06K 7/14

(52) U.S. Cl. CPC A45D 31/00; G06Q 30/0621; G06K 9/00375; G06K 9/00013; G06T 7/60; G06K 7/1408

(57) ABSTRACT

Provided are a nail measuring device, system, and method for customized nail products. The device comprises a nail gauge attached to a nail of a user to measure a three-dimensional (3D) shape data figure including a width and curvature value of the nail through photography. The device may take the form of measuring stickers which are attached to fingernails. An image is taken of fingernail having the sticker and the image is processed by the system to determine a size and shape of the nail.

### CROSS-REFERENCE TO RELATED APPLICATION

This application claims priority of Korean Patent Application No. 10-2021-0126983, filed on Sep. 27, 2021, in the KIPO (Korean Intellectual Property Office), the disclosure of which is incorporated herein entirely by reference.

### BACKGROUND OF THE INVENTION

### Field of the Invention

The present disclosure relates to a nail measuring device for customized nail products and a system and method for using the same. More particularly, the present disclosure is related to a gauge attachable to a nail to measure a three-dimensional (3D) shape including the width and curvature value of the nail via photography, and a system and method for using the same.

### Description of the Related Art

Nail art services are provided in the form of a user visiting a nail art shop to receive nail care from a professional or attaching various artificial nails to the user's own nails. Nail art services are also provided by a user purchasing a ready-made product and directly conducting a nail treatment.

When ready-made nail products are used, there may be a difference between the shape of the read-made product and the shape of the user's nail even if the product includes various sizes, because the width and the curvature of a nail varies from person to person and from finger to finger.

Because of this problem, ready-made products often do not fit the user's nail very well. In order to address this problem, a technique of measuring a three-dimensional (3D) shape of a nail by using a 3D scanner or the like as in Korea Patent Application No. 10-2011-0067945, which was published as KR 10-2011-0101106 on Sep. 15, 2011, has been proposed.

However, such a 3D scanner is expensive and may be cost prohibitive for individual users to own. It would therefore be advantageous to be able to measure nails without the drawbacks of existing systems and methods.

### SUMMARY OF THE INVENTION

Provided is a nail measuring device, system, and method for customized nail products. A device is provided for assisting a user in measuring the size and shape of a nail. An image is analyzed through an imaging device, such as a smartphone, capable of photography, to thereby measure a size and shape of the nail.

According to an aspect of the present disclosure, a nail gauge is attached to a nail of a user to measure a three-dimensional (3D) shape data figure including a width and curvature value of the nail through photography. The nail gauges comprise a pattern formed on a surface of the nail gauge.

According to an aspect of the present disclosure, a nail measuring method using a nail gauge includes an image requesting operation of requesting an image of a hand with nails to which the nail gauge is attached, an image receiving operation of receiving an image of a hand with nails to which the nail gauge is attached, a hand shape recognition operation of recognizing a hand shape from the image of the hand received in the image reception operation, a finger location recognition operation of recognizing the location of each finger from the hand shape recognized in the hand shape recognition operation, a finger image decomposition operation of decomposing an image of the nail gauge attached to each finger and matching the decomposed image with location information of each finger obtained in the finger location recognition operation, a pattern image interpretation operation of obtaining a 3D shape data figure through interpretation of a pattern image from the image of the nail gauge decomposed in the finger image decomposition operation, a width figure calculation operation of calculating a width figure by recognizing the number of patterns in a vertical direction in the pattern image interpretation operation, a width figure storage operation of storing the width figure of the nail gauge together with the location information of each finger on which the nail gauge is located through width figure information recognized in the width figure calculation operation, and a curvature calculation operation of calculating a curvature of the pattern image from the 3D shape data figure obtained in the pattern image interpretation operation and storing the curvature.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a block diagram of a nail measuring system according to an embodiment of the present disclosure.

FIG. 2 is a plan view of a nail gauge set according to an embodiment of the present disclosure.

FIG. 3 is a plan view of a nail gauge set according to an embodiment of the present disclosure.

FIG. 4 is a plan view of a nail gauge set according to an embodiment of the present disclosure.

FIG. 5 is a plan view of a nail gauge set according to an embodiment of the present disclosure.

FIG. 6 is a usage state view showing a state in which a plurality of nail gauges according to an embodiment of the present disclosure are attached onto nails.

FIG. 7 is a usage state view showing a state in which a nail gauge according to an embodiment of the present disclosure is attached onto a nail.

FIG. 8 is a plan view of a nail gauge according to an embodiment of the present disclosure.

FIG. 9 is a flowchart of a nail measuring method according to an embodiment of the present disclosure.

FIG. 10 is a view illustrating nail measurement according to an embodiment of the present disclosure.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

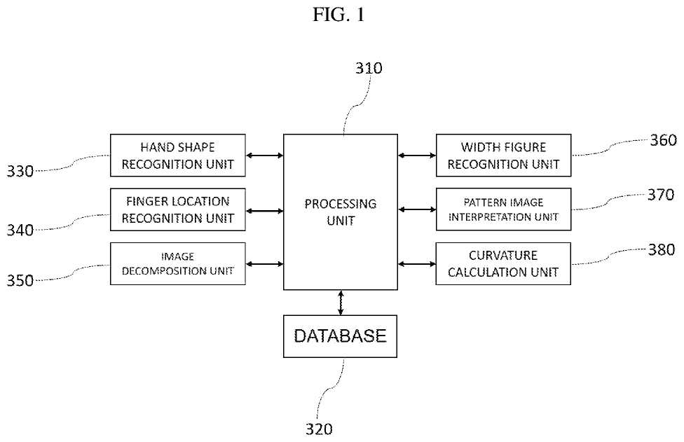

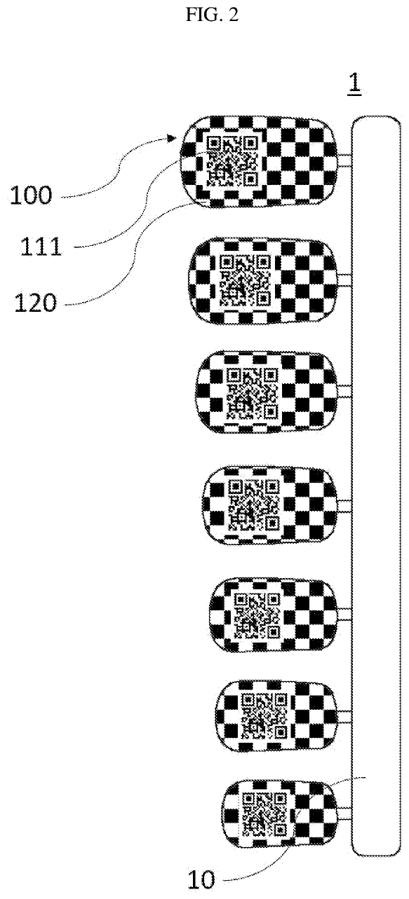

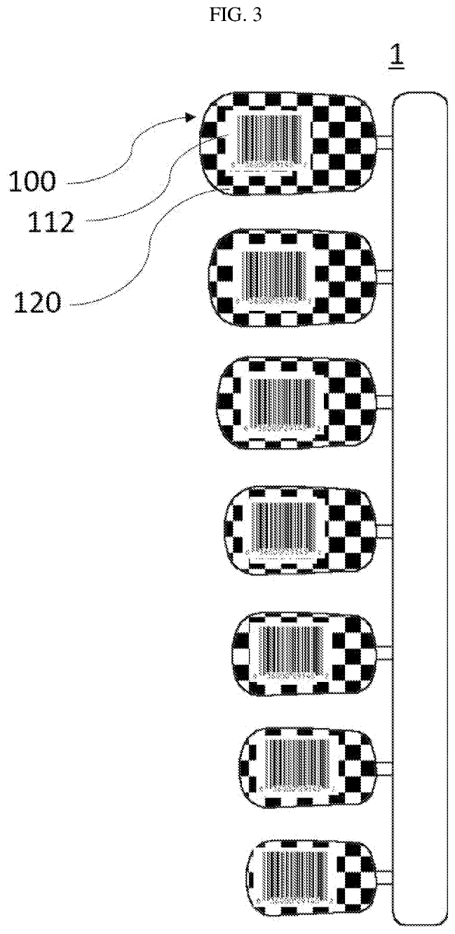

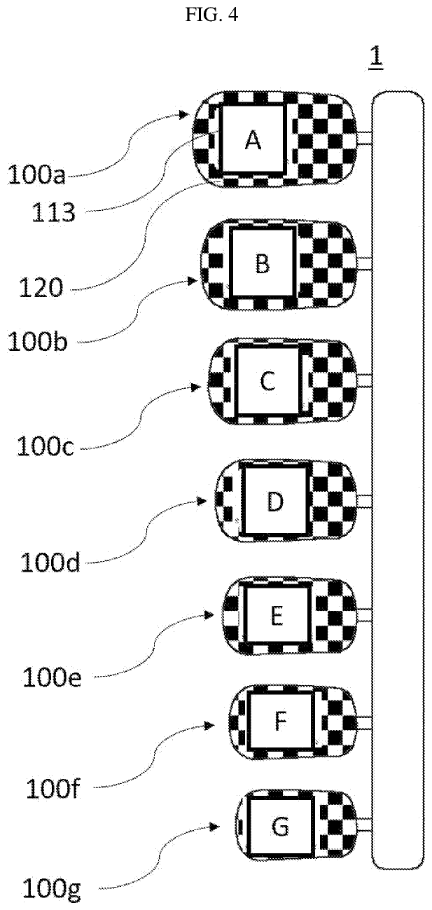

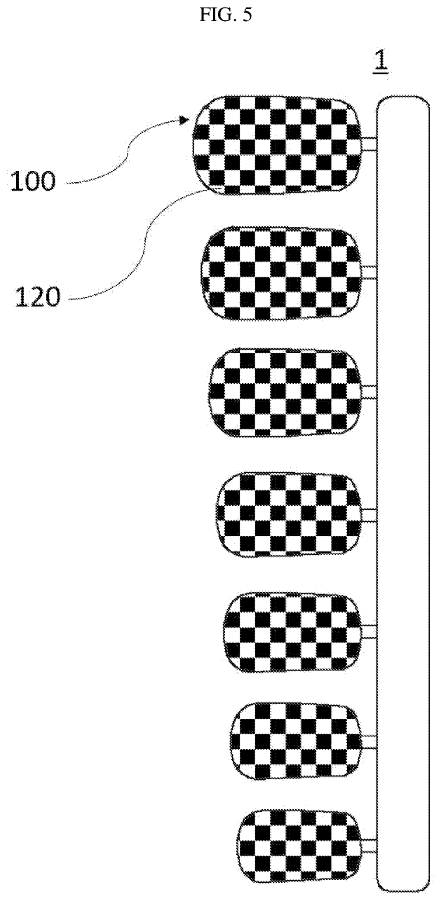

FIG. 1 is a block diagram of a nail measuring system according to an embodiment of the present disclosure. FIGS. 2 through 5 are plan views of a nail gauge set according to embodiments of the present disclosure.

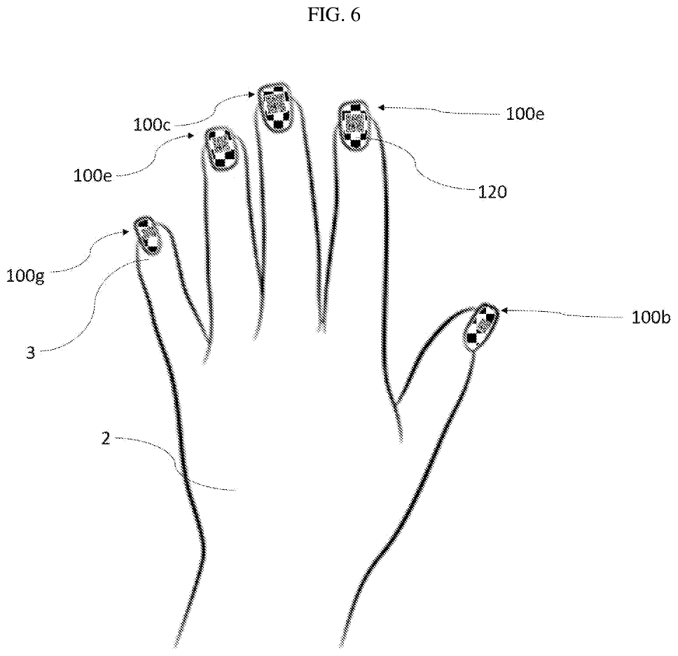

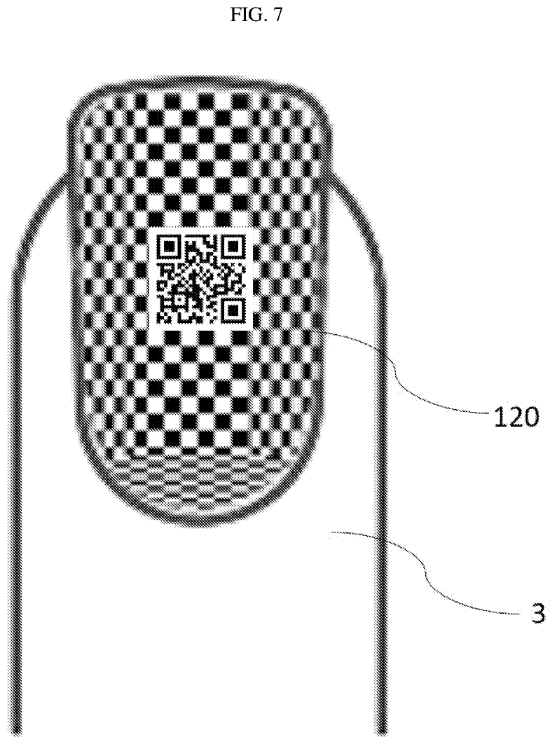

FIGS. 6 and 7 are usage state views showing a state in which a nail gauge according to an embodiment of the present disclosure is attached onto a nail.

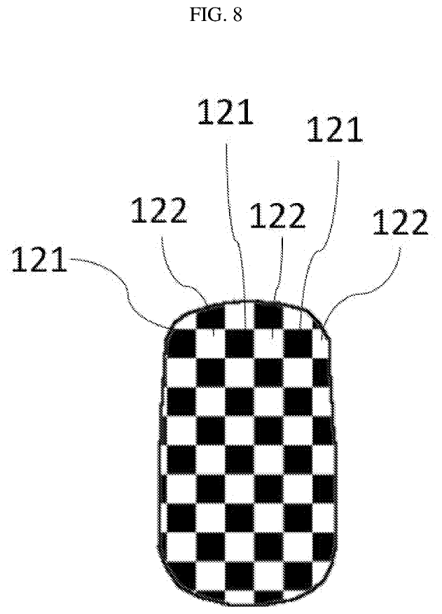

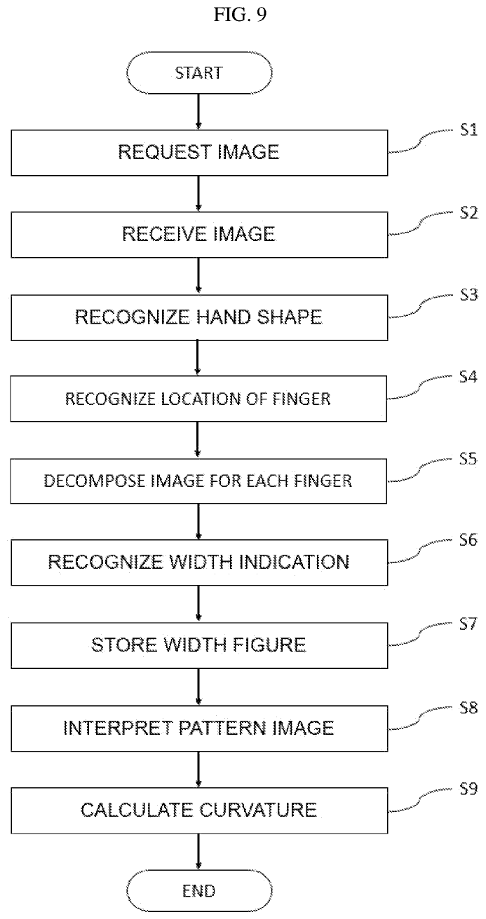

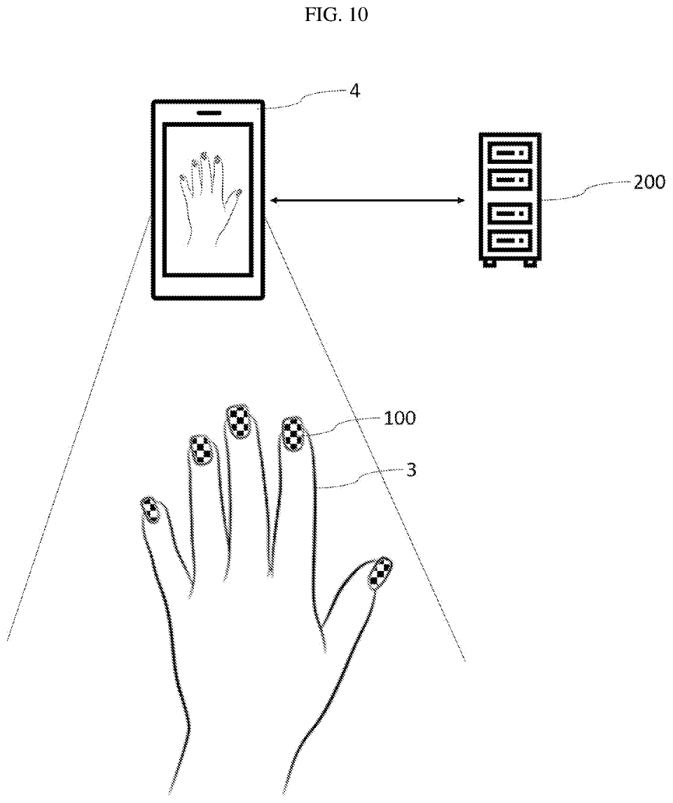

FIG. 8 is a plan view of a nail gauge according to an embodiment of the present disclosure, FIG. 9 is a flowchart of a nail measuring method according to an embodiment of the present disclosure, and FIG. 10 is a view illustrating nail measurement according to an embodiment of the present disclosure.

As shown in FIGS. 2-8, a nail gauge set 1 according to an embodiment of the present disclosure is comprised of a plurality of nail gauges 100. Each nail gauge 100 may be attached to the nail of each finger 3 of a user. A plurality of nail gauges 100 are connected to one another by a connection unit 10, so that the user may select and use nail gauges conforming to his or her nail sizes. Thus, the nail gauge set 1 includes the nail gauges 100 and the connection unit 10.

The nail gauges 100 are portions attached to the nails of the user, and a plurality of nail gauges 100 may be formed to have various sizes to correspond to various nail sizes. For example, FIGS. 2-5 show a nail gauge set comprised of 7 nail gauges. A user may select the 5 gauges which most closely match their 5 nails.

Each nail gauge 100 is comprised of a thin flexible sheet material. A repetitive pattern 120 is formed on a surface of the nail gauges 100. According to an embodiment of the present disclosure, a rectangular lattice is repeated, and, as shown in FIG. 8, a first pattern 121 and a second pattern 122 are repeated by alternating with each so that a single pattern is formed via a combination of the first pattern and the second pattern.

In this case, the pattern 120 is formed to have a constant size on the nail gauge.

In addition, width indication portions 111, 112, and 113 may be formed on the surfaces of the nail gauges, and may include a QR code, a barcode, a recognition symbol, and the like, as shown in FIGS. 2 through 4.

The nail gauge 100 may comprise an adhesive on the surface opposite the pattern. To facilitate easy removal of the nail gauge after use, the adhesive may be a removable type.

The role of the nail gauges in measuring the nails is best explained with reference to the system and method used for measuring the nails. As shown in FIGS. 1, 6, 9, and 10, a system and method using the nail gauge is shown. Nail measurement is accomplished by photographing one or more fingers 3 of a hand 2 (or the entire hand) including the nails to which the nail gauges are attached. The photograph is captured using an imaging device 4 and transmitting the image to a server 200 to process the image in the server 200 or processing the image in the imaging device 4. A system processing this method may provide a service by including a processing unit 310, a database 320 storing and outputting processed data, a hand shape recognition unit 330, a finger location recognition unit 340, an image decomposition unit 350, a width figure recognition unit 360, a pattern image interpretation unit 370, and a curvature calculation unit 380.

The nail measuring method may include the following operations.

In an image requesting operation S1, an image of a hand with nails to which the nail gauges are attached is requested. This operation is performed by the processing unit 310 of the system sending a signal to an imaging device.

In an image reception operation S2, the image of the hand with the nails to which the nail gauges are attached is received. This operation is performed by the processing unit 310 of the system receiving the image through the imaging device.

In a hand shape recognition operation S3, a hand shape is recognized from the image of the hand received in the image reception operation. This operation is performed by delivering hand shape information recognized by the hand shape recognition unit 330 having received the image of the hand from the processing unit 310 to the processing unit 310.

In a finger location recognition operation S4, the location of each finger is recognized from the hand shape recognized in the hand shape recognition operation. This operation is performed by the finger location recognition unit 340 having received the image of the hand and the hand shape information from the processing unit 310. A finger location recognized in the finger location recognition operation may be a combination of left and right information and name information that is one of thumb, index, middle, ring, and little fingers.

In a finger image decomposition operation S5, an image of the nail gauge attached to each finger is decomposed and matched with location information of each finger obtained in the finger location recognition operation. This operation is performed by the processing unit 310 and the hand shape recognition unit 330.

In a width indication portion recognition operation S6, a width indication portion is recognized and interpreted from the image of the nail gauge decomposed in the finger image decomposition operation. This operation is performed by the processing unit 310 and the width figure recognition unit 360. The shape of the width indication portion recognized in the width indication portion recognition operation is a QR code. Information included in the QR code may be matched with a length value of a horizontal width of the nail gauge set previously, and the length value of the horizontal width may be a width figure of the nail gauge.

In a width figure storage operation S7, the width figure of the nail gauge together with the location information of each finger on which the nail gauge is located is stored through information about the width indication portion recognized in the width indication portion recognition operation. This operation is performed by the processing unit 310, the width figure recognition unit 360, and the database 320.

According to another embodiment of the present disclosure, a width may be calculated through the number of patterns without using the width indication portion. In this case, the number of patterns arranged in a horizontal direction is recognized in a pattern image interpretation operation instead of the width indication portion recognition operation S6 to calculate the width, and the width is stored in the width figure storage operation S7.

In a pattern image interpretation operation S8, a 3D shape data figure is obtained through interpretation of a pattern image from the image of the nail gauge decomposed in the finger image decomposition operation. This operation is performed by the processing unit 310 and the pattern image interpretation unit 370.

In a curvature calculation operation S9, a curvature of the pattern image is calculated from the 3D shape data figure obtained in the pattern image interpretation operation and stored. This operation is performed by the processing unit 310 and the curvature calculation unit 380. The curvature calculated in the curvature calculation operation may be a left-right curvature and an up-and-down curvature. The left-right curvature is a curvature from the center of the pattern image to left and right ends of the pattern image and the up-and-down curvature is a curvature from the center of the pattern image to upper and lower ends of the pattern image.

Using the above system and method, a custom nail profile may be generated and stored for a user. The custom nail profile may include data such as identification information for the user, the date, and the data for each of the user's nails. The nail measurement for each nail may be used to select a specific nail treatment for the user. For example, a nail manufacturer may produce a large number of artificial nails having various sizes and curvatures. One or more artificial nails are selected which most closely match the size and curvature of the user's nail(s). The system may determine the artificial nail which most closely matches the user's nail in size and shape. The corresponding artificial nail(s) may be retrieved and applied at a salon or purchased by the user online or from a retail store.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A nail gauge set comprising:
a plurality of nail gauges, each nail gauge comprising:
a flexible sheet;
a repetitive pattern on one surface of the gauge; and
an adhesive on an opposing surface of the gauge from the repetitive pattern; and
a connection unit to which each nail gauge of the plurality of nail gauges are attached to.

2. The nail gauge set of claim 1, wherein a width indication portion is formed on the surface of the nail gauge comprising the repetitive pattern.

3. The nail gauge set of claim 2, wherein the width indication portion comprises a QR code.

4. The nail gauge set of claim 2, wherein the width indication portion comprises a barcode.

5. The nail gauge set of claim 2, wherein the width indication portion comprises a recognition symbol.

6. A nail measuring system comprising:
a processing unit;
a hand shape recognition unit for recognizing a hand shape from an image of a hand with nails to which a nail gauge is attached;
a finger location recognition unit for recognizing a location of each finger recognized by the hand shape recognition unit;
an image decomposition unit for decomposing an image of the nail gauge attached to each finger and matching the decomposed image with location information of each finger obtained from the finger location recognition unit;
a pattern image interpretation unit for obtaining a 3D shape data figure through interpretation of a pattern image from the image of the nail gauge decomposed by the image decomposition unit; and
a curvature calculation unit for calculating a curvature of the pattern image from the 3D shape data figure obtained by the pattern image interpretation unit,
wherein the curvature is stored by the system.

7. The nail measuring system of claim 6, further comprising:
a width figure recognition unit for calculating a width figure by recognizing a number of patterns in a vertical direction;

8. The nail measuring system of claim 6, further comprising:
a width figure recognition unit for calculating a width figure by recognizing and interpreting a width indication portion from the image of the nail gauge;

9. The nail gauge set of claim 8, wherein a width indication portion is formed on the surface of the nail gauge comprising the repetitive pattern.

10. The nail gauge set of claim 9, wherein the width indication portion comprises a QR code.

11. The nail gauge set of claim 9, wherein the width indication portion comprises a barcode.

12. The nail gauge set of claim 9, wherein the width indication portion comprises a recognition symbol.

13. A method for measuring nails, the method comprising:
an image requesting operation of requesting an image of a hand with nails to which a nail gauge is attached;
an image receiving operation of receiving an image of a hand with nails to which the nail gauge is attached;
a hand shape recognition operation of recognizing a hand shape from the image of the hand received in the image reception operation;
a finger location recognition operation of recognizing the location of each finger from the hand shape recognized in the hand shape recognition operation;
a finger image decomposition operation of decomposing an image of the nail gauge attached to each finger and matching the decomposed image with location information of each finger obtained in the finger location recognition operation;
a pattern image interpretation operation of obtaining a 3D shape data figure through interpretation of a pattern image from the image of the nail gauge decomposed in the finger image decomposition operation; and
a curvature calculation operation of calculating a curvature of the pattern image from the 3D shape data figure obtained in the pattern image interpretation operation and storing the curvature.

14. The method for measuring nails of claim 13, further comprising:
a width figure calculation operation of calculating a width figure by recognizing a number of patterns in a vertical direction in the pattern image interpretation operation; and
a width figure storage operation of storing the width figure of the nail gauge together with the location information of each finger on which the nail gauge is located through width figure information recognized in the width figure calculation operation.

15. The method for measuring nails of claim 13, further comprising:
a width indication portion recognition operation of recognizing and interpreting a width indication portion from the image of the nail gauge decomposed in the finger image decomposition operation; and
a width figure storage operation of storing the width figure of the nail gauge together with the location information of each finger on which the nail gauge is located through information about the width indication portion recognized in the width indication portion recognition operation.

16. The nail measuring service providing method of claim 15, wherein the form of the width indication portion recognized in the width indication portion recognition operation comprises a QR code, information included in the QR code is matched with a preset length value of a horizontal width of the nail gauge, and the length value of the horizontal width is a width figure of the nail gauge.

17. The nail measuring service providing method of claim 15, wherein the form of the width indication portion recognized in the width indication portion recognition operation comprises a barcode, information included in the barcode is matched with a preset length value of a horizontal width of the nail gauge, and the length value of the horizontal width is a width figure of the nail gauge.

18. The nail measuring service providing method of claim 13, wherein the curvature calculated in the curvature calculation operation is a left-right curvature, which is a curvature from a center of the pattern image to left and right ends of the pattern image.

19. The nail measuring service providing method of claim 13, wherein a finger location recognized in the finger location recognition operation is a combination of left and right information and name information that is one of thumb, index, middle, ring, and little fingers.
